(19) United States
(12) Patent Application Publication
(10) Pub. No.: US 2013/0156766 A1
(43) Pub. Date: Jun. 20, 2013

(54) TREATMENT OF DRY AGE RELATED MACULAR DEGENERATION

(71) Applicant: Allergan, Inc., Irvine, CA (US)

(72) Inventors: Jyotirmoy X. Kusari, Irvine, CA (US); Yanbin Liang, Irvine, CA (US); Larry A. Wheeler, Irvine, CA (US)

(73) Assignee: ALLERGAN, INC., Irvine, CA (US)

(21) Appl. No.: 13/677,646

(22) Filed: Nov. 15, 2012

Related U.S. Application Data
(60) Provisional application No. 61/560,118, filed on Nov. 15, 2011.

(51) Int. Cl. A61K 39/00; A61K 38/17; C07K 16/28

(52) U.S. Cl. CPC A61K 39/0005; C07K 16/2863; A61K 38/17
USPC 424/135.1; 424/139.1; 424/184.1; 514/20.8

(57) ABSTRACT

A method of treating dry age related macular degeneration (“AMD”) comprising administration to the eye of an individual in need thereof of a therapeutically effective amount of an anti-endoglin agent.

### CROSS-REFERENCE TO RELATED APPLICATION

This non-provisional patent application claims priority to U.S. provisional patent application Ser. No. 61/560,118, filed on Nov. 15, 2011, which is hereby incorporated by reference in its entirety.

### FIELD OF THE INVENTION

The present invention relates the treatment of dry age related macular degeneration, specifically with molecules that target endoglin

### BACKGROUND

Age-related macular degeneration (AMD) is the leading cause of blindness in developed nations. AMD is a medical condition which usually affects older adults and results in a loss of vision in the center of the visual field (the macula) because of damage to the retina. It occurs in “dry” and “wet” forms. It is a major cause of blindness and visual impairment in older adults (>50 years). Macular degeneration can make it difficult or impossible to read or recognize faces, although enough peripheral vision remains to allow other activities of daily life.

Neovascular or exudative AMD, the “wet” form of advanced AMD, causes vision loss due to abnormal blood vessel growth (choroidal neovascularization) in the choriocapillaris, through Bruch's membrane, ultimately leading to blood and protein leakage below the macula. Bleeding, leaking, and scarring from these blood vessels eventually cause irreversible damage to the photoreceptors and rapid vision loss if left untreated. Recently, wet AMD has been treated with anti-vascular endothelial growth factor (VEGF) agents, such as bevacizumab (trade name Avastin®), ranibizumab (trade name Lucentis®) and pegaptanib (trade name Macugen®).

It has also been suggested that endoglin (also known as CD-105 and transforming growth factor-β Receptor-III (TGFβRIII)) may be targeted instead of VEGF to stop neovascularization. See, e.g., U.S. Pat. Nos. 5,660,82 and 6,190,660, both incorporated entirely by reference. Thus, anti-endoglin agents have been proposed to treat wet AMD. See, e.g., U.S. patent application Ser. No. 12/751,907, entirely incorporated by reference.

Dry macular degeneration is a chronic eye disease that causes vision loss in the center of the field of vision. Dry macular degeneration is marked by deterioration of the macula, which is in the center of the retina—the layer of tissue on the inside back wall of the eyeball. Dry macular degeneration doesn't cause total blindness, but it worsens quality of life by blurring or causing a blind spot in the central vision. Clear central vision is necessary for reading, driving and recognizing faces. Dry AMD has three stages, all of which may occur in one or both eyes: 1) Early AMD. People with early AMD have either several small drusen or a few medium-sized drusen. At this stage, there are no symptoms and no vision loss. 2) Intermediate AMD. People with intermediate AMD have either many medium-sized drusen or one or more large drusen. Some people see a blurred spot in the center of their vision. More light may be needed for reading and other tasks. 3) Advanced Dry AMD. In addition to drusen, people with advanced dry AMD have a breakdown of light-sensitive cells and supporting tissue in the central retinal area. This breakdown can cause a blurred spot in the center of vision. Over time, the blurred spot may get bigger and darker, taking more of the central vision. People may have difficulty reading or recognizing faces until they are very close.

Because of the different underlying mechanisms between wet AMD and dry AMD, current treatments for wet AMD are not suggested or approved for dry AMD. Unfortunately, there is no medical or surgical treatment is currently available for dry AMD. Thus, there is a long felt need to a treatment for dry AMD.

### BRIEF DESCRIPTION OF THE FIGURES

FIG. 1 is a diagram of the experimental plan of a laser induced blood retinal barrier (BRB) leakage model.

FIG. 2 shows images of rat retina after laser treatment, showing an anti-endoglin antibody inhibits BRB leakage.

FIG. 3 is a diagram of the experimental plan of a blue light induced retinal degeneration model.

FIG. 4 is a chart showing retinal thickness of blue light exposed rats.

FIG. 5 is a histological image of the outer nuclear layer (ONL) of the retina of blue light exposed rats.

FIG. 6 is an image showing RPE 65 expression in rat retina.

FIG. 7 is a diagram of electroretinography of a-wave and b-wave signals in Naïve, IgG pretreated blue light exposed and anti-endoglin antibody pretreated blue light exposed rats.

### DETAILED DESCRIPTION OF THE INVENTION

Surprisingly, it has been discovered that anti-endoglin agents are protective in animal models of dry AMD and thus may be used to treat dry AMD in humans. Specifically, anti-endoglin agents were shown to be protective in laser induced blood retinal barrier (BRB) leakage and blue light induced retinal degeneration models.

Anti-endoglin agents include antibodies, anticalins (see, e.g., U.S. Pat. Nos. 7,250,297 and 7,723,476, both incorporated entirely by reference), and ankyrin repeats (see, e.g., US Patent Application Publication No. 2004/0132028, and International Patent Application Publication No. WO 02/20565, both entirely incorporated by reference), avimers (see, e.g., US Patent Application Publication Nos. 2006/0286603, 2006/0234299, 2006/0223114, 2006/0177831, 2006/0008844, 2005/0221384, 2005/0164301, 2005/0089932, 2005/0053973, 2005/0048512, 2004/0175756, all incorporated entirety by reference), nanobodies (see, e.g., U.S. Pat. No. 6,765,087 and W) 06/079372, both incorporated entirely by reference), and versabodies (see, e.g., US Patent Application Publication No. 2007/0191272, incorporated entirely by reference).

Antibodies for treatment of diseases are well known in the art. As used herein, the term “antibody” refers to a monomeric or multimeric protein comprising one or more polypeptide chains. An antibody binds specifically to an antigen (e.g. endoglin) and may be able to modulate the biological activity of the antigen. As used herein, the term “antibody” can include “full length antibody” and “antibody fragments.”

By “full length antibody” herein is meant the structure that constitutes the natural biological form of an antibody, including variable and constant regions. For example, in most mammals, including humans and mice, the full length antibody of the IgG class is a tetramer and consists of two identical pairs of two immunoglobulin chains, each pair having one light and one heavy chain, each light chain comprising immunoglobulin domains VL and CL, and each heavy chain comprising immunoglobulin domains VH, CH1 (Cg1), CH2 (Cg2), and CH3 (Cg3). In some mammals, for example in camels and llamas, IgG antibodies may consist of only two heavy chains, each heavy chain comprising a variable domain attached to the Fc region.

Antibody fragments include, but are not limited to, (i) the Fab fragment consisting of VL, VH, CL and CH1 domains, (ii) the Fd fragment consisting of the VH and CH1 domains, (iii) the Fv fragment consisting of the VL and VH domains of a single antibody; (iv) the dAb fragment (Ward et al., 1989, Nature 341:544-546) which consists of a single variable, (v) isolated CDR regions, (vi) F(ab′)2 fragments, a bivalent fragment comprising two linked Fab fragments (vii) single chain Fv molecules (scFv), wherein a VH domain and a VL domain are linked by a peptide linker which allows the two domains to associate to form an antigen binding site (Bird et al., 1988, Science 242:423-426, Huston et al., 1988, Proc. Natl. Acad. Sci. U.S.A. 85:5879-5883), (viii) bispecific single chain Fv dimers (PCT/US92/09965) and (ix) “diabodies” or “triabodies”, multivalent or multispecific fragments constructed by gene fusion (Tomlinson et. al., 2000, Methods Enzymol. 326:461-479; WO94/13804; Holliger et al., 1993, Proc. Natl. Acad. Sci. U.S.A. 90:6444-6448). In certain embodiments, antibodies are produced by recombinant DNA techniques. Other examples of antibody formats and architectures are described in Holliger & Hudson, 2006, Nature Biotechnology 23(9):1126-1136, and Carter 2006, Nature Reviews Immunology 6:343-357 and references cited therein, all expressly incorporated by reference. In additional embodiments, antibodies are produced by enzymatic or chemical cleavage of naturally occurring antibodies.

Natural antibody structural units typically comprise a tetramer. Each tetramer is typically composed of two identical pairs of polypeptide chains, each pair having one “light” (typically having a molecular weight of about 25 kDa) and one “heavy” chain (typically having a molecular weight of about 50-70 kDa). Each of the light and heavy chains are made up of two distinct regions, referred to as the variable and constant regions. For the IgG class of immunoglobulins, the heavy chain is composed of four immunoglobulin domains linked from N- to C-terminus in the order VH-CH1-CH2-CH3, referring to the heavy chain variable domain, heavy chain constant domain 1, heavy chain constant domain 2, and heavy chain constant domain 3 respectively (also referred to as VH-Cg1-Cg2-Cg3, referring to the heavy chain variable domain, constant gamma 1 domain, constant gamma 2 domain, and constant gamma 3 domain respectively). The IgG light chain is composed of two immunoglobulin domains linked from N- to C-terminus in the order VL-CL, referring to the light chain variable domain and the light chain constant domain respectively. The constant regions show less sequence diversity, and are responsible for binding a number of natural proteins to elicit important biochemical events.

The variable region of an antibody contains the antigen binding determinants of the molecule, and thus determines the specificity of an antibody for its target antigen. The variable region is so named because it is the most distinct in sequence from other antibodies within the same class. In the variable region, three loops are gathered for each of the V domains of the heavy chain and light chain to form an antigen-binding site. Each of the loops is referred to as a complementarity-determining region (hereinafter referred to as a “CDR”), in which the variation in the amino acid sequence is most significant. There are 6 CDRs total, three each per heavy and light chain, designated VH CDR1, VH CDR2, VH CDR3, VL CDR1, VL CDR2, and VL CDR3. The variable region outside of the CDRs is referred to as the framework (FR) region. Although not as diverse as the CDRs, sequence variability does occur in the FR region between different antibodies. Overall, this characteristic architecture of antibodies provides a stable scaffold (the FR region) upon which substantial antigen binding diversity (the CDRs) can be explored by the immune system to obtain specificity for a broad array of antigens. A number of high-resolution structures are available for a variety of variable region fragments from different organisms, some unbound and some in complex with antigen. Sequence and structural features of antibody variable regions are disclosed, for example, in Morea et al., 1997, Biophys Chem 68:9-16; Morea et al., 2000, Methods 20:267-279, and the conserved features of antibodies are disclosed, for example, in Maynard et al., 2000, Annu Rev Biomed Eng 2:339-376, all incorporated entirely by reference.

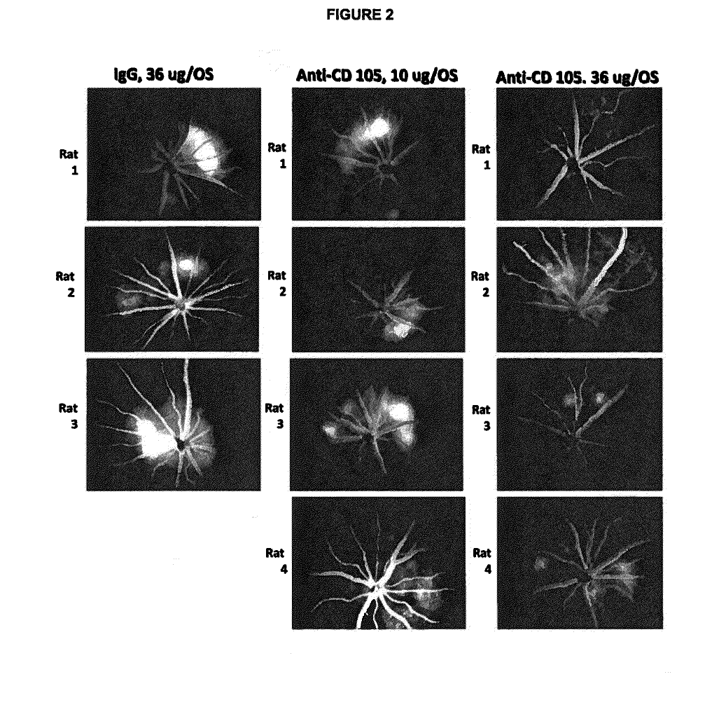

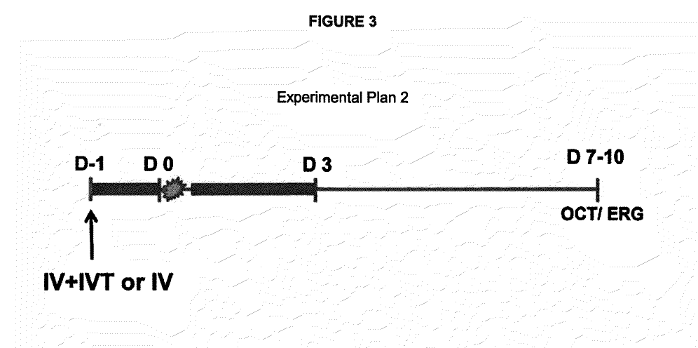

Antibodies are grouped into classes, also referred to as isotypes, as determined genetically by the constant region. Human constant light chains are classified as kappa (Ck) and lambda (CI) light chains. Heavy chains are classified as mu, delta, gamma, alpha, or epsilon, and define the antibody's isotype as IgM, IgD, IgG, IgA, and IgE, respectively. The IgG class is the most commonly used for therapeutic purposes. In humans this class comprises subclasses IgG1, IgG2, IgG3, and IgG4. In mice this class comprises subclasses IgG1, IgG2a, IgG2b, IgG3. IgM has subclasses, including, but not limited to, IgM1 and IgM2. IgA has several subclasses, including but not limited to IgA1 and IgA2. Thus, “isotype” as used herein is meant any of the classes or subclasses of immunoglobulins defined by the chemical and antigenic characteristics of their constant regions. The known human immunoglobulin isotypes are IgG1, IgG2, IgG3, IgG4, IgA1, IgA2, IgM1, IgM2, IgD, and IgE. FIG. 2 provides the sequences of the human light chain kappa and heavy chain gamma constant chains. FIG. 3 shows an alignment of the human IgG constant heavy chains.

As is well known in the art, immunoglobulin polymorphisms exist in the human population. Gm polymorphism is determined by the IGHG1, IGHG2 and IGHG3 genes which have alleles encoding allotypic antigenic determinants referred to as G1m, G2m, and G3m allotypes for markers of the human IgG1, IgG2 and IgG3 molecules (no Gm allotypes have been found on the gamma 4 chain). Markers may be classified into ‘allotypes’ and ‘isoallotypes’. These are distinguished on different serological bases dependent upon the strong sequence homologies between isotypes. Allotypes are antigenic determinants specified by allelic forms of the Ig genes. Allotypes represent slight differences in the amino acid sequences of heavy or light chains of different individuals. Even a single amino acid difference can give rise to an allotypic determinant, although in many cases there are several amino acid substitutions that have occurred. Allotypes are sequence differences between alleles of a subclass whereby the antisera recognize only the allelic differences. An isoallotype is an allele in one isotype which produces an epitope which is shared with a non-polymorphic homologous region of one or more other isotypes and because of this the antisera will react with both the relevant allotypes and the relevant homologous isotypes (Clark, 1997, IgG effector mechanisms, Chem Immunol. 65:88-110; Gorman & Clark, 1990, Semin Immunol 2(6):457-66, both incorporated entirely by reference).

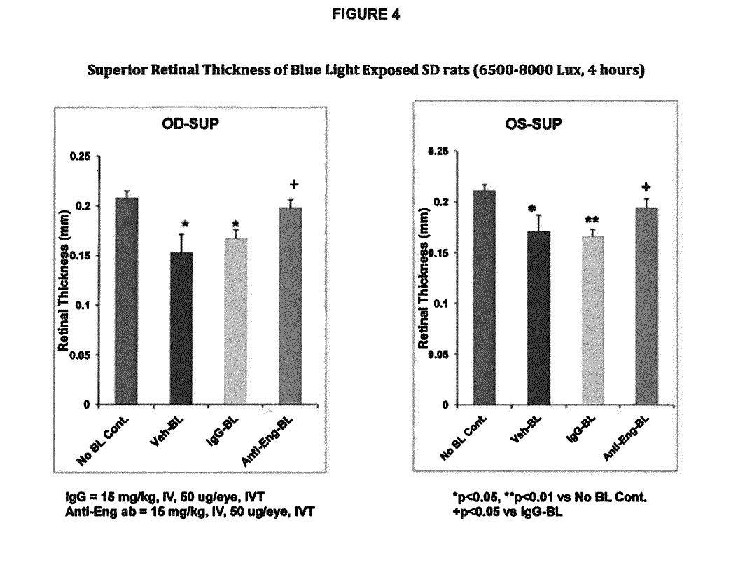

Allelic forms of human immunoglobulins have been well-characterized (WHO Review of the notation for the allotypic and related markers of human immunoglobulins. J Immunogen 1976, 3: 357-362; WHO Review of the notation for the allotypic and related markers of human immunoglobulins. 1976, Eur. J. Immunol. 6, 599-601; E. van Loghem, 1986, Allotypic markers, Monogr Allergy 19: 40-51, all incorporated entirely by reference). Additionally, other polymorphisms have been characterized (Kim et al., 2001, J. Mol. Evol. 54:1-9, incorporated entirely by reference). At present, 18 Gm allotypes are known: G1m (1, 2, 3, 17) or G1m (a, x, f, z), G2m (23) or G2m (n), G3m (5, 6, 10, 11, 13, 14, 15, 16, 21, 24, 26, 27, 28) or G3m (b1, c3, b5, b0, b3, b4, s, t, g1, c5, u, v, g5) (Lefranc, et al., The human IgG subclasses: molecular analysis of structure, function and regulation. Pergamon, Oxford, pp. 43-78 (1990); Lefranc, G. et al., 1979, Hum. Genet.: 50, 199-211, both incorporated entirely by reference). Allotypes that are inherited in fixed combinations are called Gm haplotypes. FIG. 4 shows common haplotypes of the gamma chain of human IgG1 (FIG. 4a) and IgG2 (FIG. 4b) showing the positions and the relevant amino acid substitutions. Amino acid sequences of these allotypic versions of IgG1 and IgG2 are provided as SEQ IDs: 80-85. The antibodies of the present invention may be substantially encoded by any allotype, isoallotype, or haplotype of any immunoglobulin gene.

Antibodies of the present invention may be substantially encoded by genes from any organism, including but not limited to sharks, humans, rodents including but not limited to mice and rats, lagomorpha including but not limited to rabbits and hares, camelidae including but not limited to camels, llamas, and dromedaries, and non-human primates, including but not limited to Prosimians, Platyrrhini (New World monkeys), Cercopithecoidea (Old World monkeys), and Hominoidea including the Gibbons and Lesser and Great Apes. In one embodiment, the antibodies of the present invention are substantially human or modified to look human to a human immune system. The antibodies of the present invention may be substantially encoded by immunoglobulin genes belonging to any of the antibody classes. In one embodiment, the antibodies of the present invention comprise sequences belonging to the IgG class of antibodies, including human subclasses IgG1, IgG2, IgG3, and IgG4. In an alternate embodiment, the antibodies of the present invention comprise sequences belonging to the IgA (including human subclasses IgA1 and IgA2), IgD, IgE, IgG, or IgM classes of antibodies. The antibodies of the present invention may comprise more than one protein chain. That is, the present invention may find use in an antibody that is a monomer or an oligomer, including a homo- or hetero-oligomer.

In one embodiment, the antibodies of the invention are based on human IgG sequences, and thus human IgG sequences are used as the “base” sequences against which other sequences are compared, including but not limited to sequences from other organisms, for example rodent and primate sequences, as well as sequences from other immunoglobulin classes such as IgA, IgE, IgGD, IgGM, and the like. It is contemplated that, although the antibodies of the present invention are engineered in the context of one parent antibody, the variants may be engineered in or “transferred” to the context of another, second parent antibody. This is done by determining the “equivalent” or “corresponding” residues and substitutions between the first and second antibodies, typically based on sequence or structural homology between the sequences of the two antibodies. In order to establish homology, the amino acid sequence of a first antibody outlined herein is directly compared to the sequence of a second antibody. After aligning the sequences, using one or more of the homology alignment programs known in the art (for example using conserved residues as between species), allowing for necessary insertions and deletions in order to maintain alignment (i.e., avoiding the elimination of conserved residues through arbitrary deletion and insertion), the residues equivalent to particular amino acids in the primary sequence of the first antibody are defined. Alignment of conserved residues preferably should conserve 100% of such residues. However, alignment of greater than 75% or as little as 50% of conserved residues is also adequate to define equivalent residues. Equivalent residues may also be defined by determining structural homology between a first and second antibody that is at the level of tertiary structure for antibodies whose structures have been determined. In this case, equivalent residues are defined as those for which the atomic coordinates of two or more of the main chain atoms of a particular amino acid residue of the parent or precursor (N on N, CA on CA, C on C and O on O) are within 0.13 nm and preferably 0.1 nm after alignment. Alignment is achieved after the best model has been oriented and positioned to give the maximum overlap of atomic coordinates of non-hydrogen protein atoms of the proteins. Regardless of how equivalent or corresponding residues are determined, and regardless of the identity of the parent antibody in which the antibodies are made, what is meant to be conveyed is that the antibodies discovered by the present invention may be engineered into any second parent antibody that has significant sequence or structural homology with said antibody. Thus for example, if a variant antibody is generated wherein the parent antibody is human IgG1, by using the methods described above or other methods for determining equivalent residues, said variant antibody may be engineered in a human IgG2 parent antibody, a human IgA parent antibody, a mouse IgG2a or IgG2b parent antibody, and the like. Again, as described above, the context of the parent antibody does not affect the ability to transfer the antibodies of the present invention to other parent antibodies. For example, the variant antibodies that are engineered in a human IgG1 antibody that targets one antigen epitope may be transferred into a human IgG2 antibody that targets a different antigen epitope, and so forth.

Also useful for the invention may be IgGs that are hybrid compositions of the natural human IgG isotypes. Effector functions such as ADCC, ADCP, CDC, and serum half-life differ significantly between the different classes of antibodies, including for example human IgG1, IgG2, IgG3, IgG4, IgA1, IgA2, IgD, IgE, IgG, and IgM (Michaelsen et al., 1992, Molecular Immunology, 29(3): 319-326, entirely incorporated by reference). A number of studies have explored IgG1, IgG2, IgG3, and IgG4 variants in order to investigate the determinants of the effector function differences between them. See, for example, Canfield & Morrison, 1991, J. Exp. Med. 173: 1483-1491; Chappel et al., 1991, Proc. Natl. Acad. Sci. USA 88(20): 9036-9040; Chappel et al., 1993, Journal of Biological Chemistry 268:25124-25131; Tao et al., 1991, J. Exp. Med. 173: 1025-1028; Tao et al., 1993, J. Exp. Med. 178: 661-667; Redpath et al., 1998, Human Immunology, 59, 720-727, all entirely incorporated by reference.

In the IgG class of immunoglobulins, there are several immunoglobulin domains in the heavy chain. By “immunoglobulin (Ig) domain” herein is meant a region of an immunoglobulin having a distinct tertiary structure. Of interest in the present invention are the domains of the constant heavy chain, including, the constant heavy (CH) domains and the hinge. In the context of IgG antibodies, the IgG isotypes each have three CH regions: “CH1” refers to positions 118-220, “CH2” refers to positions 237-340, and “CH3” refers to positions 341-447 according to the EU index as in Kabat. By “hinge” or “hinge region” or “antibody hinge region” or “immunoglobulin hinge region” herein is meant the flexible polypeptide comprising the amino acids between the first and second constant domains of an antibody. Structurally, the IgG CH1 domain ends at EU position 220, and the IgG CH2 domain begins at residue EU position 237. Thus for IgG the hinge is herein defined to include positions 221 (D221 in IgG1) to 236 (G236 in IgG1), wherein the numbering is according to the EU index as in Kabat. In some embodiments, for example in the context of an Fc region, the lower hinge is included, with the “lower hinge” generally referring to positions 226 or 230. The constant heavy chain, as defined herein, refers to the N-terminus of the CH1 domain to the C-terminus of the CH3 domain, thus comprising positions 118-447, wherein numbering is according to the EU index. The constant light chain comprises a single domain, and as defined herein refers to positions 108-214 of Ck or CI, wherein numbering is according to the EU index.

Antibodies of the invention may include multispecific antibodies, notably bispecific antibodies, also sometimes referred to as “diabodies”. These are antibodies that bind to two (or more) different antigens. Diabodies can be manufactured in a variety of ways known in the art, e.g., prepared chemically or from hybrid hybridomas. In one embodiment, the antibody is a minibody. Minibodies are minimized antibody-like proteins comprising a scFv joined to a CH3 domain. In some cases, the scFv can be joined to the Fc region, and may include some or all of the hinge region. For a description of multispecific antibodies see Holliger & Hudson, 2006, Nature Biotechnology 23(9):1126-1136 and references cited therein, all expressly incorporated by reference.

In one embodiment, the antibody of the invention is an antibody fragment. Of interest are antibodies that comprise Fc regions, Fc fusions, and the constant region of the heavy chain (CH1-hinge-CH2-CH3). Antibodies of the present invention may comprise Fc fragments. An Fc fragment of the present invention may comprise from 1-90% of the Fc region, with 10-90% being preferred, and 30-90% being more preferred. Thus for example, an Fc fragment of the present invention may comprise an IgG1 Cg2 domain, an IgG1 Cg2 domain and hinge region, an IgG1 Cg3 domain, and so forth. In one embodiment, an Fc fragment of the present invention additionally comprises a fusion partner, effectively making it an Fc fragment fusion. Fc fragments may or may not contain extra polypeptide sequence.

Chimeric, Humanized, and Fully Human Antibodies

Immunogenicity is the result of a complex series of responses to a substance that is perceived as foreign, and may include production of neutralizing and non-neutralizing antibodies, formation of immune complexes, complement activation, mast cell activation, inflammation, hypersensitivity responses, and anaphylaxis. Several factors can contribute to protein immunogenicity, including but not limited to protein sequence, route and frequency of administration, and patient population. Immunogenicity may limit the efficacy and safety of a protein therapeutic in multiple ways. Efficacy can be reduced directly by the formation of neutralizing antibodies. Efficacy may also be reduced indirectly, as binding to either neutralizing or non-neutralizing antibodies typically leads to rapid clearance from serum. Severe side effects and even death may occur when an immune reaction is raised. Thus in one embodiment, protein engineering is used to reduce the immunogenicity of the antibodies of the present invention.

In some embodiments, the scaffold components can be a mixture from different species. Such antibody may be a chimeric antibody and/or a humanized antibody. In general, both “chimeric antibodies” and “humanized antibodies” refer to antibodies that combine regions from more than one species. “Chimeric antibodies” traditionally comprise variable region(s) from a mouse (or rat, in some cases) and the constant region(s) from a human (Morrison et al., 1984, Proc Natl Acad Sci USA 81: 6851-6855, incorporated entirely by reference).

By “humanized” antibody as used herein is meant an antibody comprising a human framework region (FR) and one or more complementarity determining regions (CDR's) from a non-human (usually mouse or rat) antibody. The non-human antibody providing the CDR's is called the “donor” and the human immunoglobulin providing the framework is called the “acceptor”. In certain embodiments, humanization relies principally on the grafting of donor CDRs onto acceptor (human) VL and VH frameworks (Winter U.S. Pat. No. 5,225,539, incorporated entirely by reference). This strategy is referred to as “CDR grafting”. “Backmutation” of selected acceptor framework residues to the corresponding donor residues is often required to regain affinity that is lost in the initial grafted construct (U.S. Pat. No. 5,693,762, incorporated entirely by reference). The humanized antibody optimally also will comprise at least a portion of an immunoglobulin constant region, typically that of a human immunoglobulin, and thus will typically comprise a human Fc region. A variety of techniques and methods for humanizing and reshaping non-human antibodies are well known in the art (See Tsurushita & Vasquez, 2004, Humanization of Monoclonal Antibodies, Molecular Biology of B Cells, 533-545, Elsevier Science (USA), and references cited therein, all incorporated entirely by reference). Humanization or other methods of reducing the immunogenicity of nonhuman antibody variable regions may include resurfacing methods, as described for example in Roguska et al., 1994, Proc. Natl. Acad. Sci. USA 91:969-973, incorporated entirely by reference. In one embodiment, selection based methods may be employed to humanize and/or affinity mature antibody variable regions, that is, to increase the affinity of the variable region for its target antigen. Other humanization methods may involve the grafting of only parts of the CDRs, including but not limited to methods described in U.S. Ser. No. 09/810,502; Tan et al., 2002, J. Immunol. 169:1119-1125; De Pascalis et al., 2002, J. Immunol. 169:3076-3084, incorporated entirely by reference. Structure-based methods may be employed for humanization and affinity maturation, for example as described in U.S. Ser. No. 10/153,159 and related applications, all incorporated entirely by reference.

In certain variations, the immunogenicity of the antibody is reduced using a method described in U.S. Ser. No. 11/004,590, entitled “Methods of Generating Variant Proteins with Increased Host String Content and Compositions Thereof”, filed on Dec. 3, 2004, incorporated entirely by reference.

Modifications to reduce immunogenicity may include modifications that reduce binding of processed peptides derived from the parent sequence to MHC proteins. For example, amino acid modifications would be engineered such that there are no or a minimal number of immune epitopes that are predicted to bind, with high affinity, to any prevalent MHC alleles. Several methods of identifying MHC-binding epitopes in protein sequences are known in the art and may be used to score epitopes in an antibody of the present invention. See for example U.S. Ser. No. 09/903,378, U.S. Ser. No. 10/754,296, U.S. Ser. No. 11/249,692, and references cited therein, all expressly incorporated by reference.

In an alternate embodiment, the antibodies of the present invention may be fully human, that is the sequences of the antibodies are completely or substantially human. “Fully human antibody” or “complete human antibody” refers to a human antibody having the gene sequence of an antibody derived from a human chromosome with the modifications outlined herein. A number of methods are known in the art for generating fully human antibodies, including the use of transgenic mice (Bruggemann et al., 1997, Curr Opin Biotechnol 8:455-458,) or human antibody libraries coupled with selection methods (Griffiths et al., 1998, Curr Opin Biotechnol 9:102-108,) both incorporated entirely by reference.

### EXAMPLES

### Example 1

An inhibitory murine anti-endoglin antibody was developed using standard laboratory techniques.

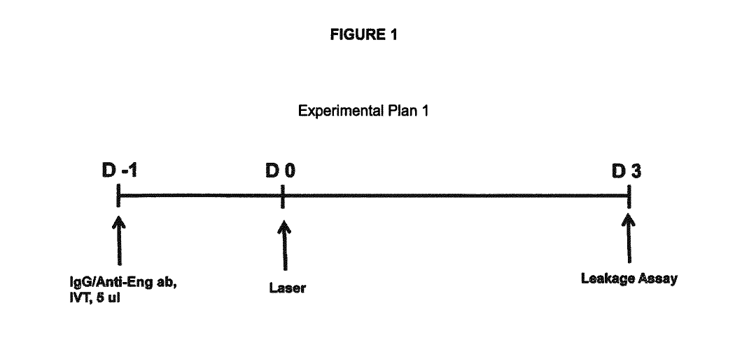

Brown Norway (BN) rats (Charles Rivers) weighing 270 to 350 grams were used in the study. BN rats were divided into three groups. One group of rats received IgG (36 μg/eye) and anti-endoglin antibody was administered to other two groups at 10 or 36 μg of antibody/eye). The antibody was delivered through intravitreal (IVT) injection (5 μl/eye) 24 hours prior to laser treatment. Argon laser (Coherent® Inc., Santa Clara Calif.) was used for photocoagulation. Left eye was selected for laser treatment for each animal, and 8 laser spots (532 nm wavelength, 500 mW power, 0.1 second duration, 100 um spot size) were concentrically delivered approximately 2 optic discs from the center while avoiding major blood vessels. BRB leakage assay was performed in rats three days after laser treatment (FIG. 1). Antibody/vehicle administration was done through Intravitreal injection on Day-1.

Laser application was performed on day 0. (Production of acute vapor bubbles at the time of laser treatment indicates rupture of the Bruch membrane.) On day 3, fundos angiograms (FA) were obtained with a Zeiss FF 450 Fundos camera coupled to a personal computer with visupack software. Anesthetized animals received intravenous injection of Fluorescein Sodium (2%, 20 mg/ml). Late-phase angiography was performed 4 to 6 minutes after the injection of sodium fluorescein

Compared to IgG, intravitreal delivery of anti-endoglin antibody inhibits laser induced BRB leakage in rats in a dose dependent fashion (FIG. 2).

At time of laser, five injury responses were observed:
BL/1=Well defined thermal bleaching (poor burn; excluded)
SBB/2=Slight Bubbling of Bruchs membrane and limited expanding thermal burn (good burn; shallow crater).
BB/3=Bubbling of Bruchs membrane and expanding thermal burn (better burn; deep crater)
BBL (petichiae)/4=Bubbling of Bruchs membrane and expanding thermal burn followed by hemorrhage (best burn; Bleeding)
H=Hemorrhage (excluded)

To compare leakage between IgG and anti-Endoglin antibody treated rats, animals with very similar burn were taken for fluorescein angiography study

### Example 2

IgG or anti-endoglin antibody was administered to Sprague Dowley (SD) rats 24 hrs before blue light exposure. The antibody was administered either by intravenous (IV) and intravitreal (IVT), or IV injection just before dark adaptation for 24 hrs. After 24 hrs, rats were exposed to blue light for 4 hrs and kept in dark adaptation room for 3 days. The rats were transferred after 3 days of dark adaptation to the room with 12 hr dark/light cycle. After 7-10 days of blue light exposure, Optical Coherence Tomography (OCT), Electroretinography (ERG) and histology studies were performed using naïve and the light treated rats (FIG. 3).

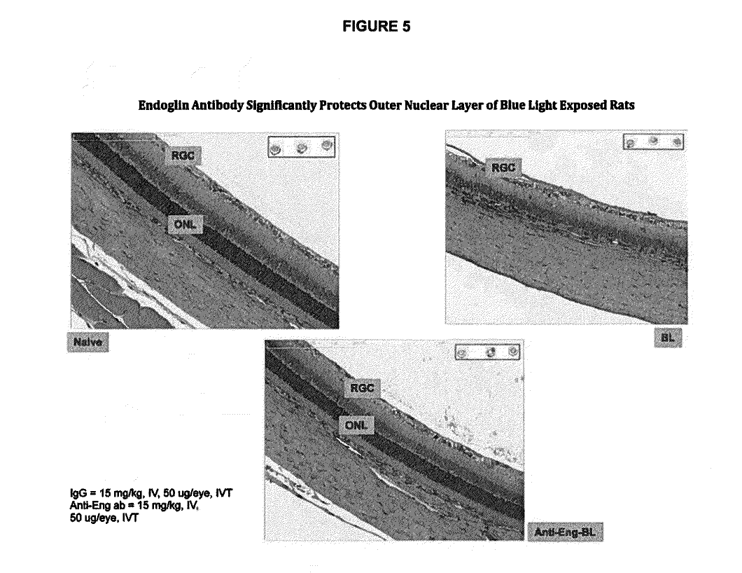

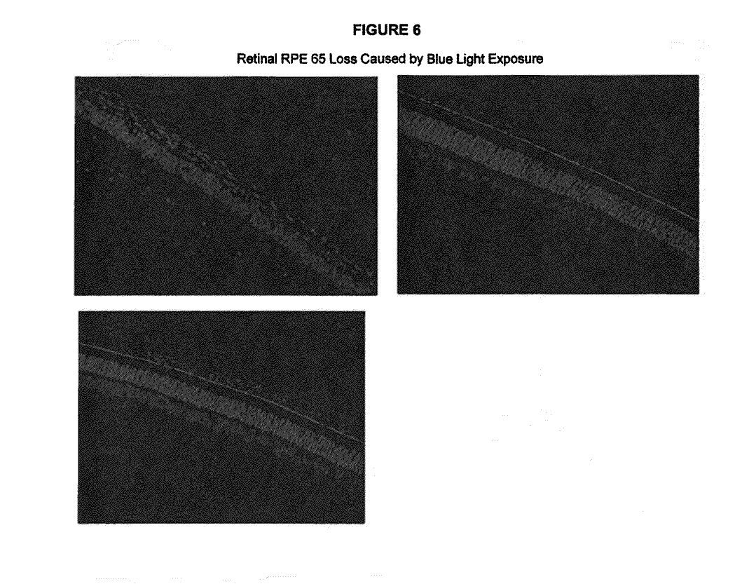

Rat Ocular Tissue Processing, H&E staining & determination of RPE65 expression: Sprague-Dawley male rats 2-3 weeks after blue light exposure were euthanized with CO2 and orbits enucleated. Eyes were fixed in Davidson's fixative overnight at room temperature and transferred to 70% ethanol for 24 hrs. Further tissue processing was done by serial dehydration in 80%, 95% & 100% alcohol and Propar, followed by paraffin embedding. Whole rat eyes were transversely cut in the vertical meridian proceeding from nasal to temporal side, using a Microtome (RM2255; Leica Microsystems). Using optic nerve head as the landmark, a total of 45 serial sections with 5 microns/section were collected on 15 glass slides. Couple of slides were diparaffinized and sequentially stained using hematoxylin (nucleus) and eosin (cytoplasm) as per standard protocol to compare photoreceptor/RPE lesion between experimental groups. The results are shown in FIG. 5. For determination of RPE65 protein expression, some of the 15 glass slides collected earlier deparaffinized and hydrated to distilled water. The slides were rinsed with distilled water: 2 changes ˜5 min each and then rinsed with PBS for 5 min. The slides were incubated in primary RPE65 antibody mixture (dil. 1:2000) for 24 hrs at 40 C and then rinsed again with PBS: 3 changes ˜10 min each. The slides were further incubated in 0.1M PBS 0.1% Triton X-100 with the secondary antibody (dil. 1:1000) and then rinsed with PBS: 3 changes ˜10 min each. The slides were finally Mounted using Prolong Gold mounting media with DAPI (FIG. 6).

Compared to no blue light control rats, blue light exposure for 4 hrs significantly reduced retinal thickness (as measured by OCT) of the exposed rats. Surprisingly, pretreatment of the rats with anti-endoglin antibody significantly prevented reduction in retinal thickness induced by blue light (FIG. 4).

In order to assess retinal morphology, retinal sections from Naïve, blue light exposed and anti-endoglin antibody pretreated blue light exposed rats were stained with hematoxylin/eosin. Compared to Naive, blue light significantly destroyed outer nuclear layer (ONL) of the retina of the exposed rats (FIG. 5). However, anti-endoglin antibody treatment prevented blue light induced ONL loss. Morphology of ONL of anti-endoglin antibody blue light exposed rats are very similar to that of Naïve animals (FIG. 5).

In order to understand Retinal Pigment Epithelial (RPE) cell status, expression of RPE cell marker-RPE 65 was examined in retina from Naïve, blue light exposed and anti-endoglin antibody pretreated blue light exposed rats using immunohistochemical approach. Compared to Naïve rats, there was no expression of RPE65 in retina of blue light exposed rats (FIG. 6). However, anti-endoglin antibody treatment preserved RPE 65 expression in retina of blue light exposed rats (FIG. 6). Thus, anti-endoglin antibody protected at least ONL and RPE cells of retina from blue light induced damage/loss.

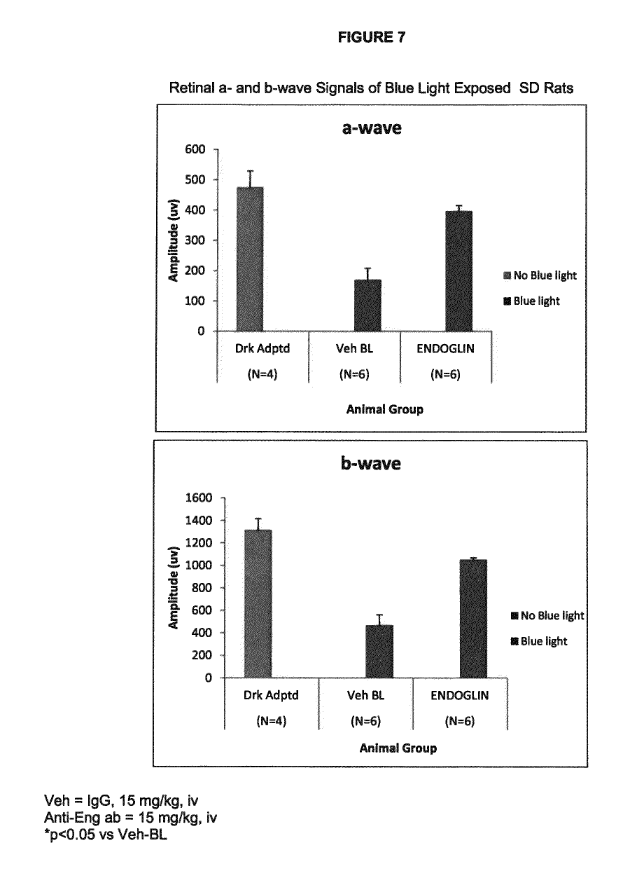

In order to assess retinal function, electroretinography (ERG) was performed in Naïve, IgG pretreated blue light exposed and anti-endoglin antibody pretreated blue light exposed rats. Compared to Naive rats, both ERG a- and b-waves were significantly compromised in blue light animals. However, anti-endoglin antibody treatment significantly protected retinal a- and b-wave signals of anti-endoglin pretreated blue light exposed rats. Amplitudes of retinal a- and b-waves of anti-endoglin antibody rats were very similar to that of Naïve rats (FIG. 7).

### Example 3

A patient with central vision blurring visits their doctor. The patent is diagnosed with dry-AMD. The doctor administers an anti-endoglin antibody by injection to the vitreous. The patient has improvement with their symptoms and the dry-AMD does not progress further.

### Example 4

A patient is diagnosed with dry-AMD. The doctor administers an anti-endoglin anticalin by injection to the vitreous. The patient has improvement with their symptoms and the dry-AMD does not progress further.

### Example 5

A patient is diagnosed with dry-AMD. The doctor administers an anti-endoglin ankyrin repeat by injection to the vitreous. The patient has improvement with their symptoms and the dry-AMD does not progress further.

### Example 6

A patient is diagnosed with dry-AMD. The doctor administers an anti-endoglin avimer by injection to the vitreous. The patient has improvement with their symptoms and the dry-AMD does not progress further.

### Example 7

A patient is diagnosed with dry-AMD. The doctor administers an anti-endoglin nanobody by injection to the vitreous. The patient has improvement with their symptoms and the dry-AMD does not progress further.

### Example 8

A patient is diagnosed with dry-AMD. The doctor administers an anti-endoglin versabody by injection to the vitreous. The patient has improvement with their symptoms and the dry-AMD does not progress further.

### Example 9

A patient is diagnosed with dry-AMD. The doctor administers an anti-endoglin biologic by injection to the vitreous. The patient has improvement with their symptoms and the dry-AMD does not progress further.

What is claimed:

1. A method of treating dry age related macular degeneration (“AMD”) comprising administration to the eye of an individual in need thereof of a therapeutically effective amount of an anti-endoglin agent.

2. The method of claim 1, wherein the anti-endoglin agent is an antibody or fragment thereof.

3. The method of claim 1, wherein the anti-endoglin agent is an anticalin.

4. The method of claim 1, wherein the anti-endoglin agent is an ankyrin repeat.

5. The method of claim 1, wherein the anti-endoglin agent is an avimer.

6. The method of claim 1, wherein the anti-endoglin agent is a nanobody.

7. The method of claim 1, wherein the anti-endoglin agent is a versabody.
